(19) United States
(12) Patent Application Publication
(10) Pub. No.: US 2015/0126876 A1
(43) Pub. Date: May 7, 2015

(54) BALLISTOCARDIOGRAM ANALYSIS METHOD AND VEHICLE BALLISTOCARDIOGRAM ANALYSIS SYSTEM USING THE SAME

(71) Applicants: Hyundai Motor Company, Seoul (KR); Kia Motors Corp., Seoul (KR); SNU R&DB Foundation, Seoul (KR); Hyundai Dymos Incorporated, Seosan-si (KR)

(72) Inventors: Byung Jae LEE, Gunpo-si (KR); Sung Jin Sah, Suwon-si (KR); Hee Chan Kim, Seoul (KR); Taek Jun Nam, Seongnam-si (KR); Tae Hyoung Yang, Seoul (KR); In Ho Lee, Hwaseong-si (KR); Chi Yul Yoon, Seoul (KR); Seung Woo Noh, Seoul (KR)

(73) Assignees: Hyundai Motor Company, Seoul (KR); Hyundai Dymos Incorporated, Seosan-si (KR); SNU R&DB Foundation, Seoul (KR); Kia Motors Corp., Seoul (KR)

(21) Appl. No.: 14/104,702

(22) Filed: Dec. 12, 2013

(30) Foreign Application Priority Data
Nov. 1, 2013 (KR) 10-2013-0132005

(51) Int. Cl. A61B 5/18; A61B 5/0205; A61B 5/00; B60W 40/08; A61B 5/11

(52) U.S. Cl. CPC A61B 5/18; B60W 40/08; A61B 5/1102; A61B 5/6893; A61B 5/7246; A61B 5/1116; A61B 5/0205; B60W 2040/0872
USPC 600/483; 600/527

(57) ABSTRACT

A BCG analysis system includes a vehicle behavior sensor, a posture measurement sensor, a pattern database, a BCG measurement unit, a signal processing unit, and a biostate recognition unit. The vehicle behavior sensor measures the physical momentum of a vehicle. The posture measurement sensor measures the posture of a testee. The pattern database stores standardized representative patterns. The BCG measurement unit measures the BCG signal of the testee. The signal processing unit obtains desired data by matching the BCG signal with a pattern of the pattern database, searching the pattern database for a new pattern when the posture of the testee and the physical momentum of the vehicle may be changed, and matching the BCG signal of the BCG measurement unit with the new pattern. The biostate recognition unit recognizes the biostate of the testee.

### CROSS-REFERENCE TO RELATED APPLICATION

The present application claims priority to Korean Patent Application No. 10-2013-0132005, filed on Nov. 1, 2013, the entire contents of which is incorporated herein for all purposes by this reference.

### BACKGROUND OF THE INVENTION

1. Field of the Invention

The present invention relates generally to a technology that is capable of measuring and analyzing the ballistocardiogram (BCG) of a driver or a passenger and taking subsequent measures, such as the performance of ventilation or the provision of a massage, using the BCG.

2. Description of the Related Art

Ballistocardiography refers to a measurement method of estimating the state of the heart by measuring a reaction when the heart pumps blood. The measurement principle of the method is to obtain a BCG signal by measuring a minimal change in acceleration or a change in body weight that is attributable to a reaction resulting from the ejection of blood by the heart.

FIG. 1 illustrates the principle of measurement of BCG in accordance with a conventional technology. In accordance with this principle, a sensor capable of measuring pressure or weight is placed between a person and a support surface and changes over time are measured. As illustrated in FIG. 1, an electrocardiogram (ECG), together with a BCG, is measured, and the BCG signal is analyzed by determining the size, time interval, inclination, etc. of the BCG based on the R peak of the ECG.

For example, FIG. 2 illustrates an example of BCG signal analysis, such as that shown in FIG. 1. In FIG. 2, I is a signal that is generated when the heart pumps blood through the main artery, and J is a signal that is generated when the blood circulates to the lower part of the body. The contractile force of the left ventricle can be analogized based on the sizes of the I and J signals.

The basic process of BCG signal analysis is to determine the location of a J peak along a time axis based on an obtained BCG waveform. A conventional method of detecting the peak includes thresholding, template matching and wavelet analysis methods. Among these methods, the template matching method is most widely used because of the characteristics of BCG signals.

In the case of the template matching method, however, when a predetermined standard template does not match a measured and received BCG signal, a serious error occurs in the detection of a peak.

In particular, in order to analyze a BCG signal using the template matching method in a special environment, such as in a vehicle in which a detected BCG signal is likely to be affected by the posture or environment of a testee, appropriate analysis and subsequent measures cannot be achieved without the appropriate modifications of the template and the adoption of counter measures.

That is, in the construction of a system for checking the bodily status of a driver or a passenger in such as way as to detect and analyze a BCG signal in a vehicle and taking appropriate measures corresponding to the checked bodily status, a sensor for detecting the BCG signal is commonly installed under a seat of the vehicle. When a BCG signal generated from the human body is transferred to the sensor, the shape of the BCG signal can be changed by influences, such as the covering of the seat and a seat cushion pad. Furthermore, the shape of the BCG signal measured by the sensor can be changed depending on the inclination of the upper body of the testee in all directions or a posture of the testee, such as the degree of slouch, because a direction along which the strongest BCG signal is generated varies depending on the direction of the heart to the surface on which the testee is seated. Accordingly, the BCG signal can be precisely analyzed by appropriately changing the template for analyzing such a change in the shape of the BCG signal depending on the posture of the testee, etc. and applying the changed template.

Furthermore, when the behavior of a vehicle suddenly changes, the posture of a driver also changes. Even in this case, a BCG signal can be precisely analyzed only when a template is appropriately changed depending on the behavior of the vehicle, etc. and then applied.

The information disclosed in this Background of the Invention section is only for enhancement of understanding of the general background of the invention and should not be taken as an acknowledgement or any form of suggestion that this information forms the prior art already known to a person skilled in the art.

### SUMMARY OF THE INVENTION

Various aspects of the present invention are directed to providing technology in which the BCG of a passenger is measured and analyzed and subsequent measures, such as performance of ventilation or provision of a massage using the BCG, are taken.

In an aspect of the present invention, a BallistoCardioGram (BCG) analysis method of measuring a BCG of a testee who is riding in a vehicle and analyzing the measured BCG, may include a vehicle measurement step of measuring physical momentum of the vehicle attributable to driving of the vehicle, a pattern search step of searching a pattern database for a pattern based on the measured physical momentum, a signal processing step of processing a signal by performing pattern matching on the measured BCG using the retrieved pattern, and a recognition step of recognizing a biostate of the testee using results of the signal processing step.

In another aspect of the present invention, a BallistoCardioGram (BCG) method of measuring a BCG of a testee who is riding in a vehicle and analyzing the measured BCG, may include a vehicle measurement step of measuring physical momentum of the vehicle attributable to the driving of the vehicle, a posture measurement step of measuring a posture of the testee, a pattern search step of searching a pattern database for a pattern based on the measured posture of the testee and the measured physical momentum of the vehicle, a signal processing step of processing a signal by performing pattern matching on the measured BCG using the retrieved pattern, and a recognition step of recognizing a biostate of the testee using results of the signal processing step.

The physical momentum of the vehicle may include steering angle, Revolutions Per Minute (RPM), driving speed, and roll angle.

The BCG analysis method may further include a posture change determination step of determining whether or not the posture of the testee and the physical momentum of the vehicle may have changed after the signal processing step, wherein when, as a result of the determination, it is determined that the posture of the testee and the physical momentum of the vehicle may have changed, a new pattern is searched for in the pattern database and then the signal processing step is performed using the new pattern.

When, as a result of the search of the pattern database, it is determined that a pattern identical with the measured posture of the testee and the measured physical momentum of vehicle is not present, a pattern generation step of generating a pattern that is most suitable for a current posture of the testee and current physical momentum of the vehicle from patterns most similarly corresponding to the current posture of the testee and the current physical momentum of the vehicle is further performed, and the signal processing step is then performed.

In further another aspect of the present invention, a BallistoCardioGram (BCG) analysis system, may include a vehicle behavior sensor configured to measure physical momentum of a vehicle, a posture measurement sensor configured to measure a posture of a testee, a pattern database configured to store representative patterns extracted by standardizing BCG signals corresponding to various postures of the testee and physical momentums of the vehicle, a BCG measurement unit configured to measure a BCG signal of the testee, a signal processing unit configured to obtain desired data by matching the BCG signal measured by the BCG measurement unit with a pattern of the pattern database, searching the pattern database for a new pattern when the posture of the testee and the physical momentum of the vehicle are changed, and matching the BCG signal of the BCG measurement unit with the new pattern, and a biostate recognition unit configured to recognize a biostate of the testee based on results of the signal processing unit.

The BCG analysis system may further include a pattern generation unit configured to generate a pattern that is most suitable for a current posture of the testee and current physical momentum of the vehicle from patterns similarly corresponding to the current posture of the testee and the current physical momentum of the vehicle, when a pattern corresponding to the posture of the testee and the physical momentum of the vehicle may have not been retrieved from the pattern database.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a diagram illustrating the principle of measurement of BCG in accordance with a conventional technology.

FIG. 2 is a diagram illustrating the analysis of a BCG signal in the prior art.

FIG. 3 is a flowchart illustrating an exemplary embodiment of a BCG analysis method according to an exemplary embodiment of the present invention.

FIG. 4 is a diagram illustrating a BCG analysis system for a vehicle according to an exemplary embodiment of the present invention.

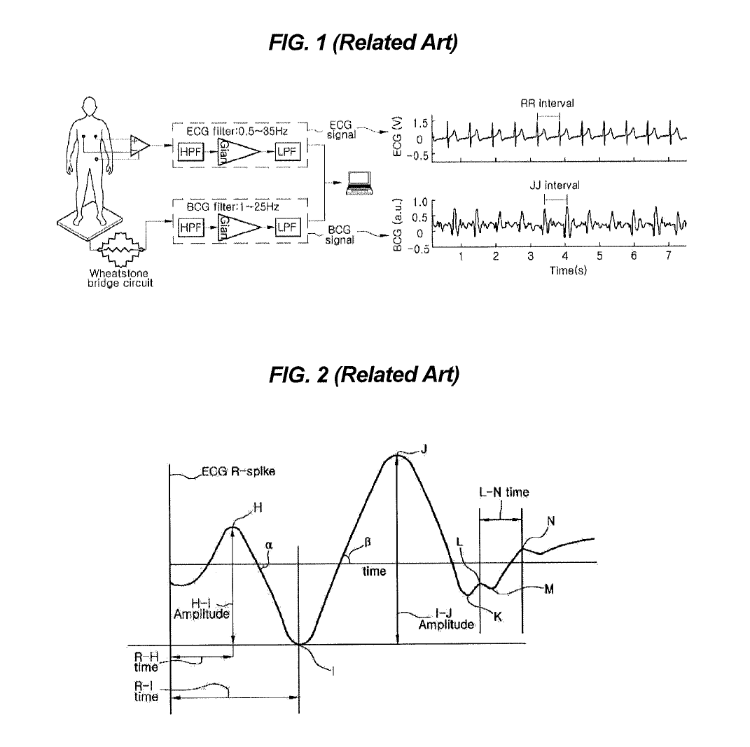

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawings and described below. While the invention(s) will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the invention as defined by the appended claims.

A BCG analysis method and the Vehicle BCG analysis system using the BCG analysis method according to embodiment of the present invention are described below with reference to the accompanying drawings.

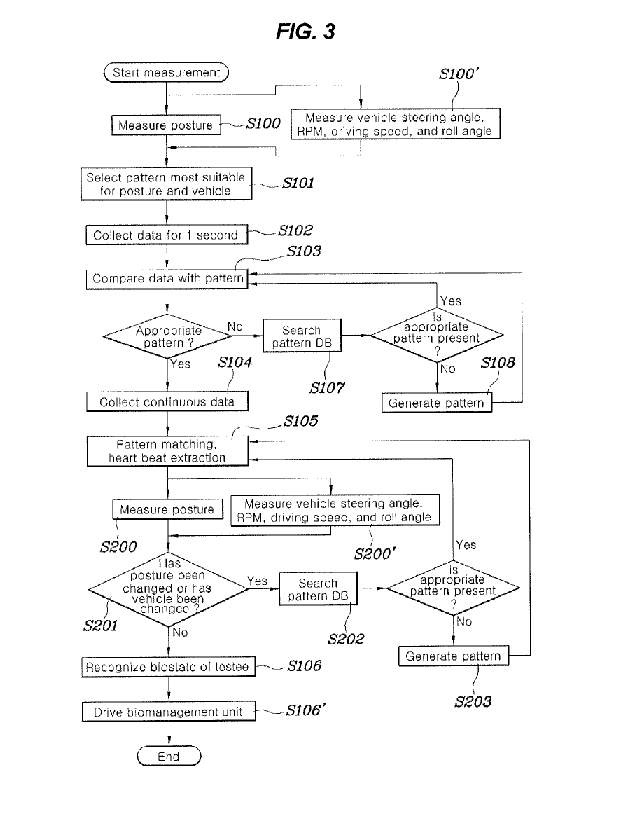

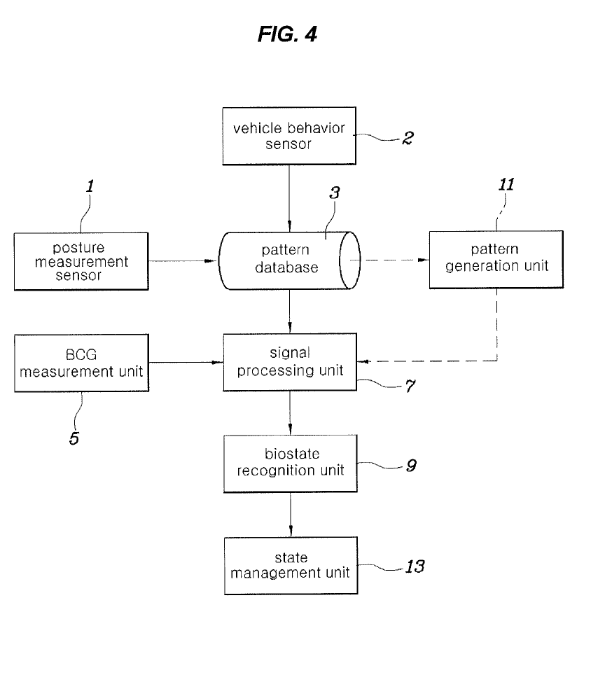

FIG. 3 is a flowchart illustrating an exemplary embodiment of a BCG analysis method according to an exemplary embodiment of the present invention, and FIG. 4 is a diagram illustrating a BCG analysis system for a vehicle according to an exemplary embodiment of the present invention.

The BCG analysis method of measuring the BCG of a testee who is riding in a vehicle and then analyzing the measured BCG according to an exemplary embodiment of the present invention includes a vehicle measurement step (S100′) of measuring the physical momentum of the vehicle attributable to the driving of the vehicle, a pattern search step (S101) of searching a pattern database for a pattern based on the measured physical momentum, a signal processing step (S105) of processing the BCG by performing pattern matching on the measured BCG using the retrieved pattern, and a recognition step (S106) of recognizing the biostate of the testee using the results of the signal processing step.

More particularly, referring to FIG. 3, the BCG analysis method of measuring the BCG of a testee who is riding in a vehicle and then analyzing the measured BCG according to an exemplary embodiment of the present invention includes a posture measurement step (S100) of measuring the posture of the testee, a vehicle measurement step (S100′) of measuring the physical momentum of the vehicle attributable to the driving of the vehicle, a pattern search step (S101) of searching a pattern database for a pattern based on the measured posture and the measured physical momentum of the vehicle, a signal processing step (S105) of performing signal processing by performing pattern matching on the measured BCG using the retrieved pattern, and a recognition step (S106) of recognizing the biostate of the testee using the results of the signal processing step.

That is, since the pattern search step (S101) is performed based on the posture of the testee measured in the posture measurement step (S100) and the behavior state of the vehicle measured in the vehicle measurement step (S100′), the signal processing step (S105) can be performed by always performing appropriate pattern matching regardless of the posture of the testee and a change in the behavior of the vehicle. Accordingly, BCG can be analyzed more rapidly and precisely.

A posture change determination step (S201) of determining whether or not the posture of the testee has changed is further performed after the signal processing step (S105). If, as a result of the determination, it is determined that the posture of the testee and the physical momentum of the vehicle have changed, a new pattern is searched for in the pattern database, and the signal processing step (S105) is performed using the new pattern.

Therefore, a BCG can be always analyzed precisely and rapidly by continuously monitoring the posture of the testee and the behavior of the vehicle and incorporating changes in the posture of the testee and the behavior of the vehicle into the BCG in real time. Accordingly, responsiveness and accuracy in the provision of various services, such as the performance of ventilation or the provision of a massage, using the analyzed BCG, can be achieved.

If, as a result of the search of the pattern database, it is determined that a pattern corresponding to the measured posture of the testee and the physical momentum of the vehicle is not present, a pattern generation step (S108) of generating a pattern that is most suitable for the current posture of the testee and the current physical momentum of the vehicle from patterns most similarly corresponding to the current posture of the testee and the current physical momentum of the vehicle may be performed, and then the signal processing step (S105) may then be performed.

That is, patterns corresponding to all postures of a testee and all vehicle behaviors may not be stored in the pattern database. Thus, if a posture or vehicle behavior is measured as ambiguous and cannot be determined to be a posture or a vehicle corresponding to a specific pattern, a new pattern suitable for the measured posture or physical momentum is generated and applied based on patterns corresponding to a posture or vehicle behavior that is similar to the ambiguous posture or vehicle behavior. For example, if a vehicle suddenly goes over irregular terrain, suddenly stops or suddenly starts while a driver is sitting in an upright posture, the BCG signal suddenly changes, and the BCG signal may become obscured by noise. In such a case, patterns most similarly corresponding to such a vehicle behavior may be fetched, and a new pattern for the case where an upright seating posture and an abnormal vehicle behavior occur at the same time can be generated by combining the fetched patterns.

The exemplary embodiment of FIG. 3 is described in more detail below. When a BCG of the testee starts being measured, a basic pattern is selected at step S101. The data of the measured BCG is measured for a specific time at step S 102. Whether or not a basic pattern is an appropriate pattern is determined by comparing the measured data with the basic pattern at step S103. If, as a result of the determination, it is determined that the basic pattern is an appropriate pattern, continuous BCG data is collected at step S104. Signal processing based on pattern matching is performed using the continuous data at step S105. The biostate of the testee is recognized based on the results of the signal processing at step S106. The posture of the testee and physical momentum of the vehicle are measured at steps S200 and S200′. If the posture or the vehicle behavior has changed at step S201, the basic pattern changes into an appropriate pattern retrieved from the pattern database, and the signal processing is performed at step S202.

That is, when the measurement of BCG is started, a basic pattern is selected, BCG data is measured and collected for the specific time, for example, for 1 second, and whether or not the basic pattern is an appropriate pattern is determined by comparing the measured and collected BCG data with the pattern. If, as a result of the determination, it is determined that the basic pattern is an appropriate pattern, continuous BCG data is collected, pattern matching using the appropriate pattern and signal processing for extracting other pieces of necessary information are performed on the continuous BCG data. The biostate of the testee, such as stress or an arousal state, is recognized using the results of the signal processing.

If, as a result of the determination, it is determined that the basic pattern is not an appropriate pattern, however, an appropriate pattern is searched for in the pattern database at step S107. If, as a result of the searching, a pattern suitable for the posture of the testee is not found, a pattern that is most suitable for the current posture of the testee is generated from patterns corresponding to a posture that is similar to the current posture of the testee at step S108.

The representative physical momentum of the vehicle for searching may include steering angle, Revolutions Per Minute (RPM), driving speed, and roll angle.

A BCG analysis system for implementing the BCG analysis method of the present invention is described below with reference to FIG. 4. The BCG analysis system includes a vehicle behavior sensor 2 for measuring the physical momentum of a vehicle, a posture measurement sensor 1 for measuring the posture of a testee, a pattern database 3 for storing representative patterns extracted by standardizing BCG signals corresponding to various postures of the testee and physical momentum of the vehicle, respectively, a BCG measurement unit 5 for measuring the BCG signal of the testee, a signal processing unit 7 for obtaining desired data by matching the BCG signal measured by the BCG measurement unit with a pattern of the pattern database, searching the pattern database for a new pattern if the posture of the testee and the physical momentum of the vehicle change, and matching the BCG signal of the BCG measurement unit with the new pattern, and a biostate recognition unit 9 for recognizing the biostate of the testee based on the results of the signal processing unit.

The BCG analysis system may further include a pattern generation unit 11 for generating a pattern that is most suitable for a current posture of the testee and current physical momentum of the vehicle from patterns similarly corresponding to the current posture of the testee and the current physical momentum of the vehicle, if a pattern corresponding to the posture of the testee and the physical momentum of the vehicle has not been retrieved from the pattern database.

A camera for photographing a testee, an inertia sensor attached to the body, an inclination sensor installed in a seat, an elasticity sensor installed in a safety belt, and a pressure sensor placed in a seat may be used as the posture measurement sensor 1 selectively or in combination. A movement of the center of gravity of a testee, inclinations in all directions, and the degree of slouch are checked using the sensors.

Furthermore, various types of conventional vehicle sensors capable of measuring steering angle, RPM, driving speed, and roll angle, that is, physical momentum of the vehicle, may be used as the vehicle behavior sensors.

The BCG analysis system may further include a state management unit 13 for driving elements of the vehicle which can affect the biostate of the testee depending on the biostate recognized by the biostate recognition unit 9.

That is, if the state management unit 13 is added to the BCG analysis system of the present invention, the state management unit 13 may be a display device for displaying the biostate of a testee so that the testee can view the biostate, an audio alarm device for generating an alarm announcement, a ventilation device, or a massage device that is included in a seat.

In accordance with the above-described BCG analysis method and the above-described vehicle BCG analysis system using the method, a BCG signal that is measured from a testee is appropriately analyzed by appropriately taking changes in the posture of the testee into consideration in an environment, such as in a vehicle. Accordingly, the bodily state of the testee can be precisely diagnosed, and the marketability of a vehicle can be maximized by taking appropriate and subsequent measures, such as providing a testee with a massage function or interior ventilation in a vehicle, based on the diagnosed bodily state.

In particular, even when the behavior of a vehicle severely changes due to a sudden stop, a quick start, or an accident, the distortion of measured data can be prevented by analyzing a BCG signal while considering such conditions.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. A BallistoCardioGram (BCG) analysis method of measuring a BCG of a testee who is riding in a vehicle and analyzing the measured BCG, comprising:
a vehicle measurement step of measuring physical momentum of the vehicle attributable to driving of the vehicle;
a pattern search step of searching a pattern database for a pattern based on the measured physical momentum;
a signal processing step of processing a signal by performing pattern matching on the measured BCG using the retrieved pattern; and
a recognition step of recognizing a biostate of the testee using results of the signal processing step.

2. A BallistoCardioGram (BCG) method of measuring a BCG of a testee who is riding in a vehicle and analyzing the measured BCG, comprising:
a vehicle measurement step of measuring physical momentum of the vehicle attributable to the driving of the vehicle;
a posture measurement step of measuring a posture of the testee;
a pattern search step of searching a pattern database for a pattern based on the measured posture of the testee and the measured physical momentum of the vehicle;
a signal processing step of processing a signal by performing pattern matching on the measured BCG using the retrieved pattern; and
a recognition step of recognizing a biostate of the testee using results of the signal processing step.

3. The BCG analysis method of claim 2, wherein the physical momentum of the vehicle comprises steering angle, Revolutions Per Minute (RPM), driving speed, and roll angle.

4. The BCG analysis method of claim 2, further comprising a posture change determination step of determining whether or not the posture of the testee and the physical momentum of the vehicle have changed after the signal processing step, wherein when, as a result of the determination, it is determined that the posture of the testee and the physical momentum of the vehicle have changed, a new pattern is searched for in the pattern database and then the signal processing step is performed using the new pattern.

5. The BCG analysis method of claim 2, wherein when, as a result of the search of the pattern database, it is determined that a pattern identical with the measured posture of the testee and the measured physical momentum of vehicle is not present, a pattern generation step of generating a pattern that is most suitable for a current posture of the testee and current physical momentum of the vehicle from patterns most similarly corresponding to the current posture of the testee and the current physical momentum of the vehicle is further performed, and the signal processing step is then performed.

6. A BallistoCardioGram (BCG) analysis system, comprising:
a vehicle behavior sensor configured to measure physical momentum of a vehicle;
a posture measurement sensor configured to measure a posture of a testee;
a pattern database configured to store representative patterns extracted by standardizing BCG signals corresponding to various postures of the testee and physical momentums of the vehicle;
a BCG measurement unit configured to measure a BCG signal of the testee;
a signal processing unit configured to obtain desired data by matching the BCG signal measured by the BCG measurement unit with a pattern of the pattern database, searching the pattern database for a new pattern when the posture of the testee and the physical momentum of the vehicle are changed, and matching the BCG signal of the BCG measurement unit with the new pattern; and
a biostate recognition unit configured to recognize a biostate of the testee based on results of the signal processing unit.

7. The BCG analysis system of claim 6, further comprising a pattern generation unit configured to generate a pattern that is most suitable for a current posture of the testee and current physical momentum of the vehicle from patterns similarly corresponding to the current posture of the testee and the current physical momentum of the vehicle, when a pattern corresponding to the posture of the testee and the physical momentum of the vehicle has not been retrieved from the pattern database.
